(19) United States
(12) Patent Application Publication
(10) Pub. No.: US 2007/0167819 A1
(43) Pub. Date: Jul. 19, 2007

(54) Method for in-vivo measurement of biomechanical properties of internal tissues

(76) Inventors: Thomas Ward Osborn III, Cincinnati, OH (US); Hyundae Hong, West Chester, OH (US); Donna Rene Hill, Verona, KY (US); John David Norcom, Middletown, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/295,280

(22) Filed: Dec. 6, 2005

(51) Int. Cl. A61B 8/14

(52) U.S. Cl. 600/462

(57) ABSTRACT

A method for determining material properties of internal tissues of a body, the method comprising the steps of: a. providing force measuring device, b. providing an imaging means for acquiring images of deformation or displacement of tissues inside a body cavity; c. providing a calculation means for quantifying displacement of internal tissues; d. inserting at least a portion of said probe head into the body cavity; e. applying force with said probe head against an internal body tissue; f. measuring said force applied to said internal body tissue; g. acquiring images of displacement of said internal body tissue; h. calculating said displacement of said internal body tissue; and i. calculating material properties from said measured force and said calculated displacement.

### FIELD OF THE INVENTION

This invention relates to the measurement and determination of biomechanical properties of internal tissues or organs of a living body, such as a human body.

### BACKGROUND

Understanding the biomechanical properties of biological tissues, particularly internal tissues or organs of a human, is essential for the development of improved medical diagnostic and treatment tools. In addition, understanding the biomechanical properties such as the elastic and viscoelastic (i.e., strain rate dependent mechanical behaviors) properties of internal tissues or organs can aid in designing safer, more comfortable and effective devices for application on the in vivo condition. Biomechanical implications learned from these measurements can improve not only the design of medical devices and implants used for minimally invasive surgery, but also any other products interacting with body tissues. As an example, knowledge of biomechanical properties can help in developing a better understanding of the effects of internally worn devices such as tampons on the deformations in internal tissues to the point of affecting comfort and effectiveness of the devices.

External tissues and organs such as the stratum corneum and epidermis can be relatively easily characterized for in vivo mechanical properties because of easy accessibility and locating the point of measurement. However, internal tissues and organs, such as intra-abdominal tissues, intra-vaginal tissues, intra-uterine tissues, intra-esophageal tissues, and the likes are more difficult to characterize. In particular, in-vivo measurements of internal tissues to obtain biomechanical properties are difficult due to limited accessibility nature of such tissues and difficulties associated with locating the point of measurement. The constraints of available devices and techniques to reach these tissues, as well as the difficulty of obtaining accurate data under in vivo condition has hampered efforts at accurately modeling of ‘living’ internal tissue biomechanical properties.

In-vivo measurements of internal tissues properties of organs such as the vagina are particularly difficult to achieve because of limited accessibility for direct measurement of tissues and complex mechanical interactions of surrounding tissues and organs. The human female vagina is located in the lower pelvic cavity and surrounded by the major organs such as the uterus, the bladder, and the rectum. The vagina is a collapsed tube-like structure composed of fibromuscular tissue layers. The central portion has an H-shaped cross section and its walls are suspended and attached to the paravaginal connective tissues. The vaginal inner walls have rugal folding which is extended significantly during delivery, which implies hyperelastic properties. Smooth muscle fibers are oriented along the vaginal axis and arranged circularly toward the periphery. Vaginal walls are connected to the lateral pelvic floor by connective tissues and smooth muscle layers, which allow the vagina deformed and displaced easily according to the external strain energy applied.

The pelvic environment comprises a soft tissue and muscle “hammock” to which the various organs are attached. For example, the vagina is connected to the pelvis by the pelvic floor muscles and connective tissue. Because of it location within pelvic cavity, the degree of vaginal tissue deformation is significantly influenced by the biomechanical properties of surrounding organs and tissues. Furthermore, because there is no rigid supporting structure around the vagina, but connective tissues of smooth muscle fibers among the surrounding organs, it is important to understand not only deformation of vaginal tissues, but also surrounding organs' boundaries for complete measurement of biomechanical properties and parameters of vaginal and surrounding tissues. Among the surrounding organs of vagina, the bladder is the most influential organ in a way that the vaginal tissue responds to external strain; as the bladder expands by accumulating urine, it stretches toward vesicovaginal tissue layers. The apparent physical change is deformation (stretching and/or compaction) of tissue layers, which can in turn impact the stiffness of tissue layers. Interactions among the lower pelvic floor organs make the in vivo measurement of vaginal tissue even more challenging work. Therefore, these anatomical complexities of the vagina and surrounding tissues and organs require that biomechanical properties be obtained by considering the heterogeneous and inhomogeneous nature of the related human anatomy, and interactions of neighboring organs and tissues.

Accordingly, there is a continuing unaddressed need for better devices and methods for determining biomechanical properties of internal tissues and organs.

Further, there is a continuing unaddressed clinical need for devices and methods for measuring biomechanical tissue properties in-vivo, such that the effects of surrounding tissues and organs are taken into account.

Additionally, there is a continuing unaddressed need for a device and method for determining the biomechanical properties of different portions of the same tissue or organ.

Furthermore, there is a continuing unaddressed need for devices and methods for determining the biomechanical properties of internal tissues such that they can be described to include complex anatomical and mechanical factors such as viscoelasticity, hyperelasticity, heterogeneity, and directionality.

### BRIEF DESCRIPTION OF THE DRAWINGS

FIG. 1 is a side view of a device of one embodiment of a device of the present invention.

FIG. 2 is a side view of a device of one embodiment of a device of the present invention showing a partial cross-sectional representation of Section 2-2 of FIG. 1.

FIG. 3 is a cross-sectional representation of one embodiment of a probe head of the present invention.

FIG. 4 is a schematic flow representation of a method used in the present invention, together with corresponding ultrasound images.

FIGS. 5 (a)(c) are a series of representative contour maps.

FIG. 6 is a flow diagram showing a portion of the process of the present invention.

FIG. 7 is a series of graphical representations of output from the method of the present invention.

### SUMMARY OF THE INVENTION

A method for determining material properties of internal tissues of a body, the method comprising the steps of:
a. providing force measuring device, said force measuring device comprising:
   i. a probe head having a dimension suitable for insertion into the body cavity;
   ii. a load cell operatively connected to said probe head such that forces on said probe head are transmitted to said load cell;
   iii. a relatively rigid portion joined to said probe head by a relatively flexible portion such that said probe head is flexibly joined to said relatively rigid portion and can move laterally with respect to a longitudinal center line of said relatively rigid portion; and
b. providing an imaging means for acquiring images of deformation or displacement of tissues inside a body cavity;
c. providing a calculation means for quantifying displacement of internal tissues;
d. inserting at least a portion of said probe head into the body cavity;
e. applying force with said probe head against an internal body tissue;
f. measuring said force applied to said internal body tissue;
g. acquiring images of displacement of said internal body tissue;
h. calculating said displacement of said internal body tissue; and
i. calculating material properties from said measured force and said calculated displacement.

### DETAILED DESCRIPTION OF THE INVENTION

The method and device of the present invention overcomes the technical challenges and problems associated with determining in vivo the biomechanical properties of tissues. In particular, the method and device of the present invention can be used to determine location-dependent biomechanical properties, i.e., properties that are specific to a particular location in the body and/or on a particular tissue. The method and device of the present invention can include a measurement system in a combined format of a strain gauge type physiological pressure transducer to measure the tissue loading stress, and imaging devices such as a CT, a magnetic resonance imaging (MRI), or an ultrasound imager to measure localized tissue deflection and strain profiles. Such imaging devices permit non-invasive, externally disposed probes to be utilized for the purpose of making measurements of static or dynamic tissue deformation. The method of the present invention also comprises modeling internal tissues of a body by numerical methods, including finite element analysis to reconstruct tissue and organ models based on biomechanical properties measured with the present invention.

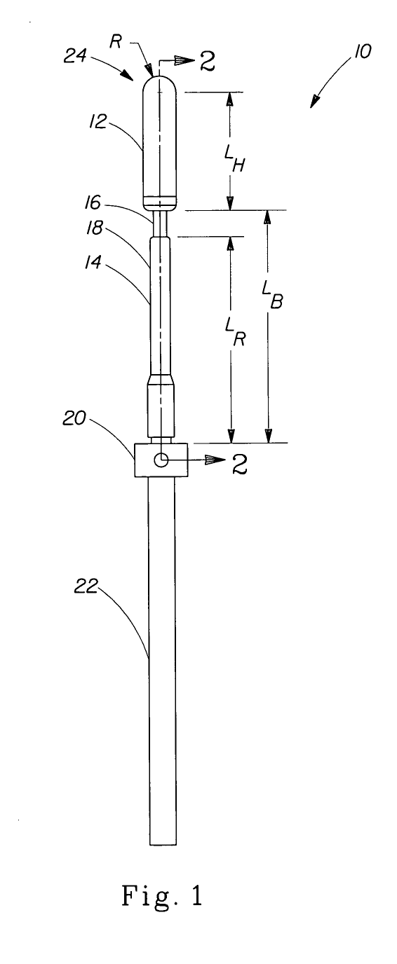

A device of the present invention is shown in FIG. 1, which shows a device 10 of the present invention that can be used to determine biomechanical properties of internal tissues of a body which can be a human or an animal. The device 10 can be used to measure biomechanical properties inside the vagina of a female mammal, including a female human. However, the device 10 can be used to determine biomechanical properties of any internal tissues and organs that can be accessed through body orifices sufficiently large for insertion of the internally-disposed portions of the device. The internal tissues can be in a body cavity, including a vagina, a vaginal opening, an anus, a urethral opening, a urethra, an auricular opening, a nasal opening, and an oral opening.

The device 10 of the present invention can be used to measure stress and strain of internal tissues, and can also be used to measure device insertion and removal force that could be indicative of device/tissue interaction and tissue shearing properties. The device 10 can be termed a “probe,” and includes at least four main parts: a probe head 12, a probe body 14 including a flexible connecting portion 16, and a relatively rigid housing 18, a load cell 20, and a handle portion 22. As shown below, load cell 20 can be located within the probe head 12.

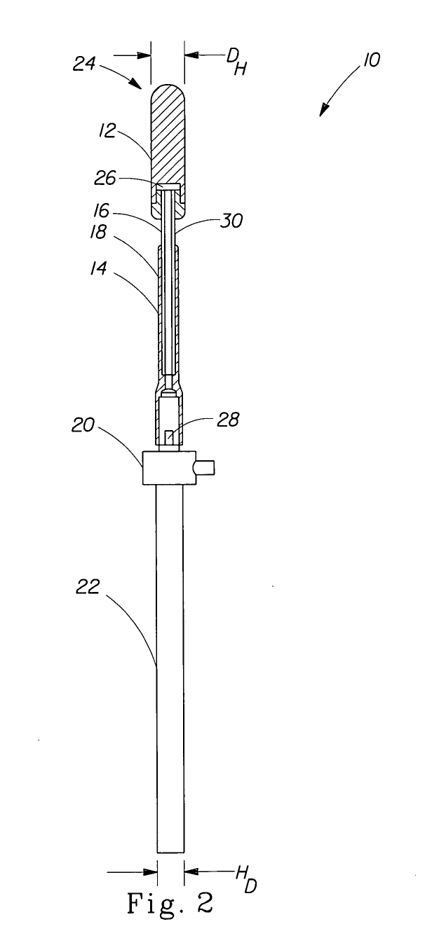

Probe head 12 is designed to be inserted into body cavities and contact internal tissues. In one embodiment probe head 12 can have a generally circular cylindrical shape, having a generally cylindrically circular cross-section. As shown in FIG. 2, probe head 12 can have an outside diameter D_{H} that can be constant and can range from about 10 mm to about 25 mm, with the limitation being to not exceed the diameter of the body cavity into which the probe is to be inserted. It is contemplated that probe head can have a diameter of as low as 2 mm to about 4 mm. Probe head 12 can have a maximum diameter D_{H} of about 14 mm (about 0.5512 inch) for use in a human vagina. Probe head 12 can have a length L_{H} determined by the constraints of the body cavity in which it is to be inserted. For use in a human vagina, the probe head 12 can have a length from a proximal end to the start of start of the radius of curvature at the distal end (as shown in FIG. 1) of from about 20 mm to about 80 mm. In one embodiment, probe head 12 length L_{H} measured to the start of the radius of curvature at the distal end 24 is about 40 mm (about 1.57 inches). Probe head radius of curvature R at the distal end 24 can be one half of the probe head diameter D_{H}.

Probe head 12 can be made of any material that is not detrimental to the body tissues it may contact. Probe head can be made of generally smooth, molded, plastic having a shape conducive to being positioned inside body cavities without causing tissue damage or discomfort to a subject. Moldable materials can include polymeric materials such as polyolefins, polyesters, polyamides, and any other generally rigid, impact resistant plastic materials. In general, as discussed below with respect to use of ultrasound imaging to aid in data collection, probe head 12 can be made of a material chosen to have a specific gravity close to that of human tissue, i.e., about 1.0. By choosing a material having a specific gravity close to 1.0, probe head 12 does not unduly degrade the ultrasound signal by attenuation. The ultrasound signal can be tuned to image human tissue, therefore, a probe head 12 having a specific gravity other than that of human tissue tends to attenuate the ultrasound signal differently, which can result in poor image quality posterior to the path of ultrasound waves through an organ of interest. In one embodiment, probe head 12 is made of high impact polystyrene (HiPS) because its specific gravity of 1.04 is very close to that of human tissue. Without being bound by theory, it is believed that a probe head 12 having a specific gravity from about 0.98 to about 1.2 can yield sufficiently clear image quality for the use of ultrasound imaging.

Probe head 12 can be connected to a load cell 20 by a probe body 14 can have a relatively rigid housing 18 and can be connected to probe head 12 by a relatively flexible connecting portion 16. The probe body 14 can have a length L_{B} sufficient to permit the probe head 12 to extend into a body cavity and contact internal tissues. In general, the length L_{R} of relatively rigid portion 18 can be from about 40 mm to about 200 mm and the length of flexible connecting portion 16 can be from about 20 to about 40 mm. In one embodiment, the relatively rigid portion 18 can be about 80 mm and the flexible connecting portion 16 can be about 20 mm, for a total length L_{B} of probe body 14 of about 100 mm.

Flexible connecting portion 16 can comprise a flexible solid rod or flexible hollow tube 30 and can function as a 360° swivel to allow the probe head 12 to deflect laterally in any direction. In one embodiment a flexible plastic tube 30 extends through the relatively rigid portion 18 to join probe head 12 at probe connection 26 to load cell 20 at load cell connection 28. Connections may require additional mechanical coupling parts, such as threaded connectors, and can be made by any means known in the art, including threaded connection, adhesive connection, frictional force fit-butt coupling, and the like. Connections can be made such that very little energy is absorbed or lost at the connections, thereby ensuring more accurate force transmission to the load cell 20. Therefore, force is transmitted from probe head 12 to load cell 20 via the flexible tube 30, a portion of which extends internally to relatively rigid portion 18, as shown in FIG. 2.

Flexible tube 30 can be made of plastic, and the stiffness of tube 30 can be varied by choice of polymer as well as choice of wall thickness. In one embodiment suitable for use in a human vagina, a nylon tube having an outside diameter of from 3 mm to about 8 mm and inside diameter of from about 2 mm to about 7 mm can be used.

Attached to the probe body 14 in a manner to receive forces transmitted from probe head 12 via flexible tube 30 is a load cell 20. Load cell 20 can be any of known strain-gauge type load cells known in the art of a suitable size to be incorporated in a hand held instrument such as device 10. Load cell 20 can have internal diaphragms to increase the cross-stiffening qualities to reduce the effect of cross loading and help assure more accurate reading of directional loading along the major axis (i.e., the longitudinal length axis) of the device 10. Suitable load cells can be obtained from Entran as one of the ELFS Series, such as the ELFS-B1 load cell incorporating a full Wheatstone bridge with semiconductor sensing elements, and which can measure up to 100 N (20 pounds). Another example is the Entran ELPS Series, such as the ELPS-T1 which can measure up to 25 N (5 pounds).

Load cell 20 measures compressive load force data during use of device 10 and can transmit the force data via a shielded cable to data collection means, such as computerized data collection module and programs.

A handle portion 22 as shown in FIG. 1 is joined to load cell 20. Joining can be by means known in the art, including threaded connection, adhesive connection, frictional force fit, and the like. An Entran ELPS-T1 load cell, for example, comprises opposing threaded connections such that flexible tube 30 can be joined either directly or indirectly on one side of the load cell. In one embodiment, flexible tube 30 is joined to rigid portion 18, which is joined to load cell 20 on one side, and handle portion 22 is joined to load cell 20 on its other side, to have a configuration as shown in FIGS. 1 and 2.

Device 10 can maintain a generally straight, longitudinally-oriented configuration, as shown in FIG. 1. However, in some embodiments, handle portion 22 can be joined by a flexible joint such as a universal, or “U-joint” to have added flexibility in use. Handle portion 22 can have any suitable diameter or size H_{D}, from about 5 mm to about 25 mm. In one embodiment, handle diameter H_{D} can be from about 8 mm to about 12 mm.

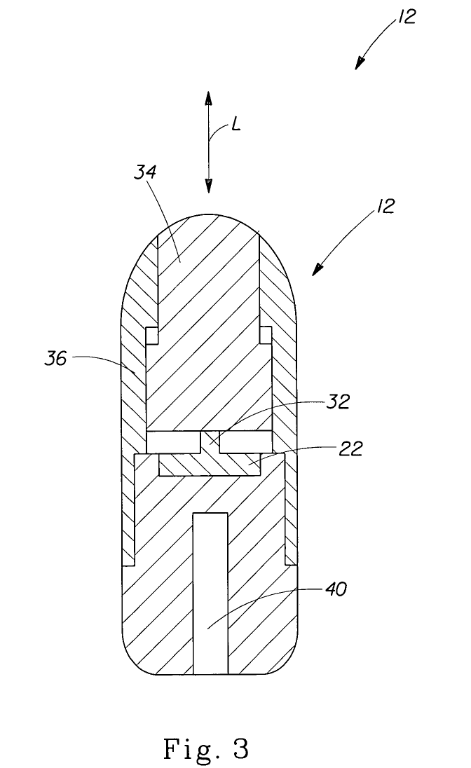

In another embodiment, load cell 22 can be positioned inside probe head 12. As shown in FIG. 3, load cell 22 can be mounted such that a biased portion 32 of load cell 22 is biased against a slideable member 34 inside probe head housing 36, the slideable member being free to move in at least two directions in the longitudinal dimension L shown. Slideable member 34, therefore, can function to transfer force from the tip of probe head 12 to load cell 22, which load cell can be electrically connected to transmit load data by means well known in the art. For example, wiring can be disposed to run through flexible tube 30 and through handle portion 22. Flexible tube 30 can be joined to probe head 12 in tube connection cavity 40. Biasing of load cell 22 to slideable member 34 can be by any means known in the art, including spring means to spring load the load cell 22 against the slideable member 34 with a constant force that is zeroed out prior to calculating load on the load cell.

Device 10 can be used to measure insertion and removal force of a device having substantially the same size or material characteristics of probe head 12. Device 10 can also be used to measure force and pressure exerted on a tissue. Device 10 can be used by inserting probe head 12 into the desired body cavity and removing it, or, optionally, by inserting and manipulating by hand via handle portion 22. For example, probe head 12 can be inserted into the vaginal opening and positioned to put pressure on internal tissues to measure tissue deflection and applied load at locations such as against the bladder or against the cervix. Tissue modulus values can also be calculated by coupling the device 10 force measurement output with a device capable of measuring strain. In one embodiment an ultrasound device can be used to measure tissue strain, as discussed below.

The method of the present invention generally involves the use of device 10 to affect pressure changes and rates of change of pressure while the strain and rates of strain changes of tissues and/or organs is detected and measured via tissue imaging means, such as medical ultrasound imagers. Pressure can be measured directly via a pressure sensor fitted to the thread instead of force load cell 20 while the imaging device measures tissue strain by measuring changes in position or changes in dimensions of tissues or organs. The pressure signal is evaluated to estimate the loading stress applied on a defined in-vivo area, thereby later enabling the calculation of material parameters such as modulus of tissues and/or organs. Such a device is useful, for example, for determining tissue properties required for modeling the insertion, expansion, and pressure application of a device penetrating the vaginal orifice, such as a tampon inserted into a vagina.

### Method of Use

In one embodiment, the method of use for measuring insertion and removal forces includes inserting the device 10 into a body cavity of interest and/or removing it and measuring the forces registered by load cell 20. In one embodiment forces can be recorded in a time versus force graph, and maximum forces can be measured and recorded. For example, probe head 12 can be sized to simulate a tampon or a tampon applicator, and device 10 can be used to measure insertion, deployment, and/or removal forces of the tampon or applicator within the vaginal path.

In another embodiment, the method of use includes inserting the device 10 into a body cavity of interest, utilizing an imaging means to detect dimensional changes at the area of interest, and correlating the measured force parameters with measured strain parameters to determine biomechanical properties of internal tissues and/or organs such as a modulus of elasticity. For example, probe head 12 can be sized to simulate a tampon or a tampon applicator, and device 10 can be used to measure insertion or deployment force within the vaginal path and corresponding tissue deflection. An ultrasound device can detect and record dimensional changes, tissue deflection, and other spatial changes such as internal organ movement. This data can be used to better understand and build a computational model of lower pelvic floor biomechanics. Each boundary layer of interest (i.e., in a computer model such as FEA analysis) such as the bladder wall, vesicovaginal connective tissue, or rectovaginal tissue, can be characterized for mechanical properties such as elastic modulus.

The location of the probe head 12 can be verified by utilizing an ultrasound imaging means, for example an ultrasound imaging means used with ultrasound B mode. In one embodiment, the ultrasound probe can be a Voluson 730®) Abdominal Transducer, Model RAB4-8, operated at about 560 micron resolution. In addition to verifying the location of probe head 12, the ultrasound imager can detect and record the corresponding position of tissue boundaries. Thus, for example, in addition to imaging the probe head 12 and a portion of the vagina, ultrasound imager can image the bladder wall, a portion of the uterus, cervix, and some of the rectovaginal tissue layers.

As probe head 12 contacts and deforms adjacent tissue layers, imaging means can detect and record deflection, displacement, deformation, or other changes in tissues or organs. In one embodiment, ultrasound imaging device can be used in M-mode during the insertion or positioning of probe head 12. While permitting higher quality of tissue motion profile, the M-mode only works at certain scanning paths, i.e., one-dimensional paths for a one-dimensional scanning profile. In another embodiment, B-mode based strain analysis can be used. Most ultrasound imagers have video mode (Cine mode) of image recording, therefore, analysis of time dependent tissue deformation is also possible.

Imaging means can capture information about tissue strain and/or tissue strain rate. Net tissue displacement can be determined as well as net displacement or deformation of tissue boundaries and adjacent organs. In particular, B-mode imaging can be efficiently utilized to determine net tissue deformation while M-mode imaging can be efficiently utilized to calculate dynamic tissue strain. Furthermore, using Cine operation of B-mode of a typical medical ultrasound imager such as the Voluson 730® from GE Healthcare (Waukesha, Wis.), it is possible to acquire time dependent tissue deflection profiles with proper image analysis. This method can be useful for the measurement of creep phenomena of vaginal tissue layer, for example.

The device and method of the present invention facilitates measurement of both insertion/deployment force of the probe or products such as a tampon within the vaginal path and corresponding tissue deflection phenomena. Data obtained by the method can provide understanding of the mechanical interaction between the vaginal tissue layers and insertion products like tampons or tampon applicators. Those data can also provide important understanding of lower pelvic floor biomechanics and each boundary layer of interest, such as the inferior bladder wall, the vesicovaginal connective tissue, the rectovaginal connective tissue, and cervix.

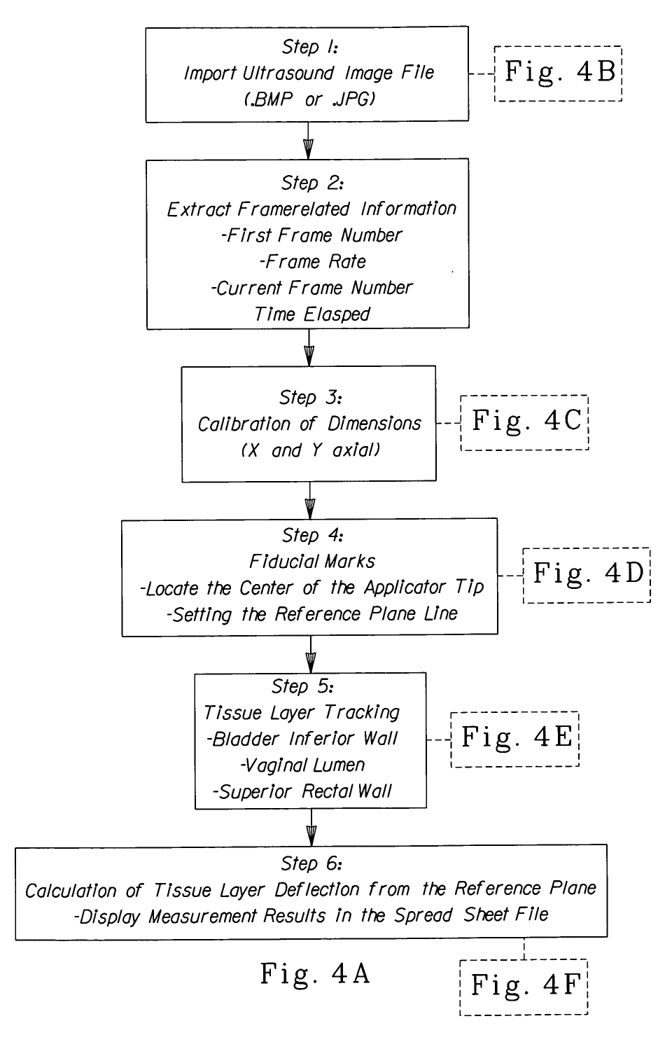

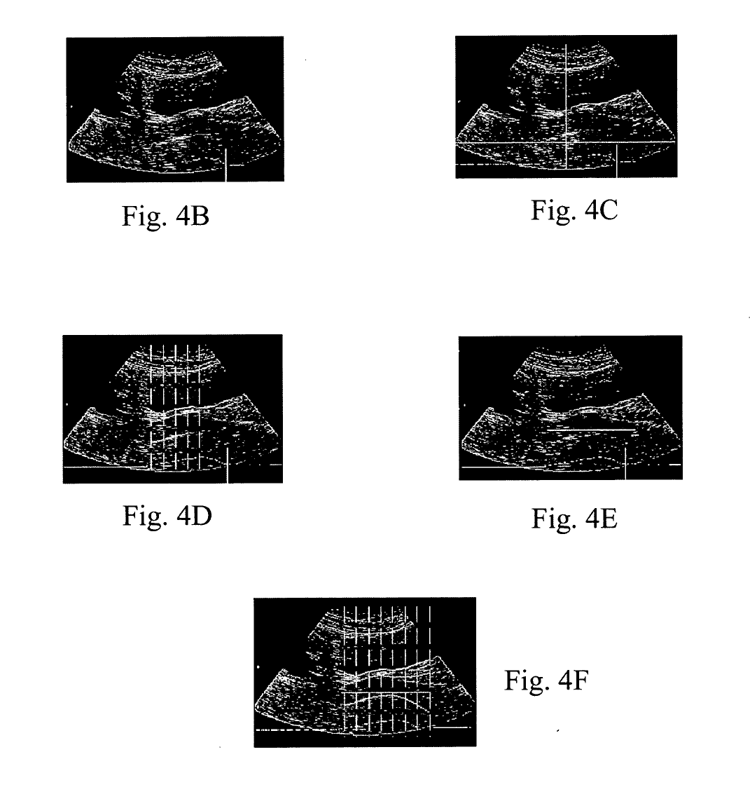

The method of the present invention can be understood with respect to the flow diagram of FIG. 4. FIG. 4 shows the functional diagram of image analysis of the corresponding ultrasound images captured by a ultrasound imager such as Voluson 730® that can provide both B- and M-mode images on the same screen, if necessary, to help in locating the measurement positions where to monitor the tissue motion profile. The same method can be reapplied on other types of imaging modality such as MRI. Image processing can be carried out with any suitable image processing tool or programming languages, such as Matlab (Mathworks, Natick, Mass.), by any means known in the art.

Insertion and/or positioning of device 10 can be recorded with ultrasound video recording mode-Cine mode. Step one of the processes for analyzing internal organs or tissue includes acquiring discrete video frames from the ultrasound video images by use of image processing programs, such as Matlab Image Processing Tool Box®. The discrete images produced in step one can then be arranged in consecutive order in a temporal arrangement of video frames. In step two the frame rate and frame number of each image can be used to calculate the total time elapsed during the insertion/positioning process started.

Because original ultrasound images may not provide geometric scaling factors for tissue deflection measurement, to make a calibration in step three, the Matlab program compares the image domain scales (X and Y axial pixels) of the video frames with actual anatomical dimensions provided by ultrasound imager to obtain calibration factors along the horizontal (X axis) and vertical (Y axis). Also, because the Voluson 730® provides gauge tools to measure any length of interested structure within the sector scanned image, the best measurement accuracy is feasible when the calibration is done along the two orthogonal directions. Therefore, in Step 3 of FIG. 4, the program calculates the conversion factor along the X and Y directions using the number of voxels involved in those two directions. With this calibration process, measurement of any length along any direction within the image can be made.

In step four, the Matlab program determines and coordinates fiducial points, which in the case of vaginal imaging can be two end points making a reference line along the vaginal lumen. From this reference line, all the tissue deflection measurements can be made. The Matlab program sets the beginning and end points of measurement along the reference line, which in the case of vaginal imaging can include the inferior boundary of a bladder wall. Then the Matlab program divides the reference line by equally spaced measurement portions, which as shown in step five of FIG. 4, can include multiple (in FIG. 4, seven as an example) measurement positions. The Matlab program can then draws vertical lines associated with the measurement portions and connect crossing points between those lines and major organ boundaries such as bladder inferior wall, vesicovaginal connective tissue, and rectovaginal connective tissue. Once the measurements are achieved over the crossing points of interest, the data collected can be saved in a data file as spread sheet format (Step 6).

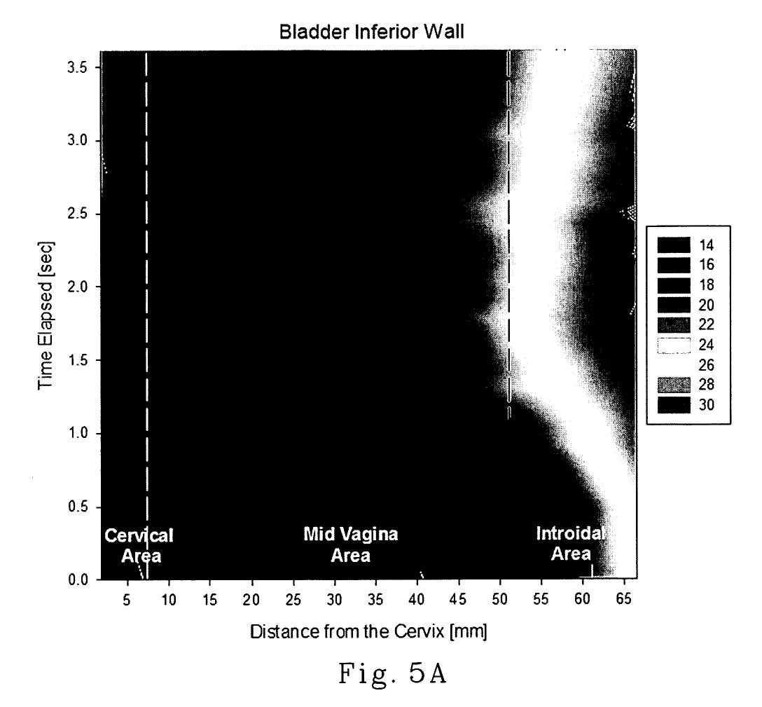

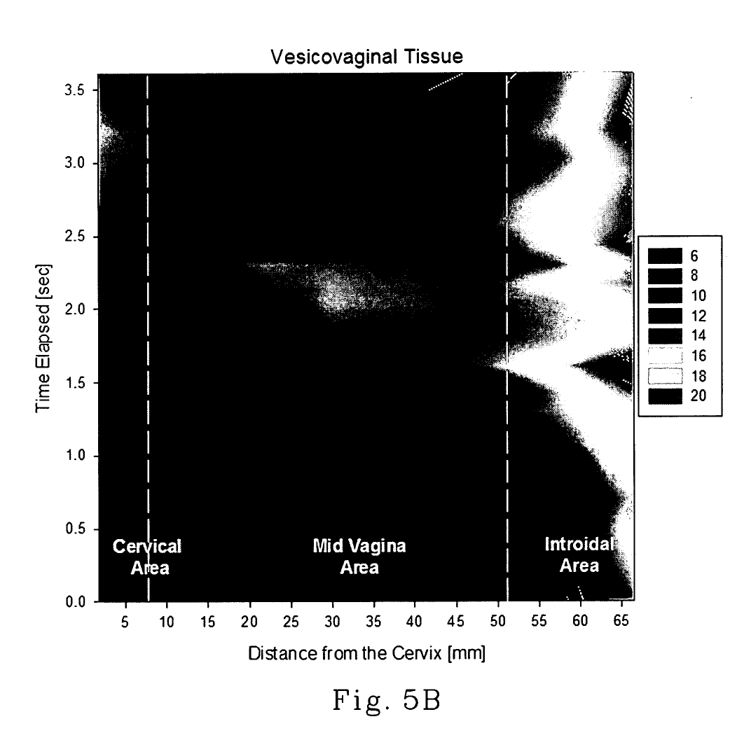

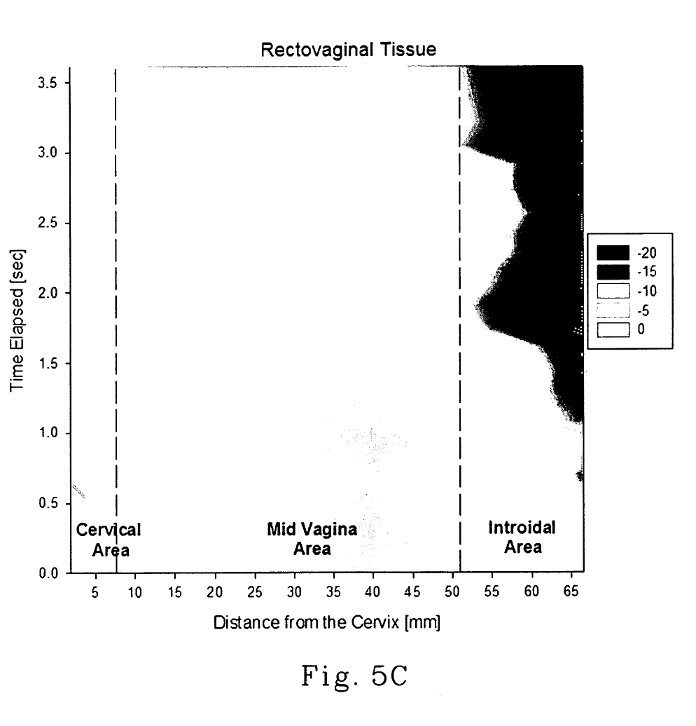

Based on the analysis described above with respect to the flow diagram of FIG. 4, calculation of tissue deflection measurement can be any visualization technique. One embodiment of post processing is shown in each of the contour maps of FIGS. 5(a)-(c). The three contour maps show the tissue deflection profiles measured along different measuring points associated with three different tissue boundaries (step 5 of the process described above). The time origin (t=0.0 sec) in each plot is the moment when a noticeable change in the tissue layer geometry occurred due to the contact with device 10. This estimation of time zero is based on an ultrasound B mode sagittal view. The three contour maps of FIG. 5 correspond to the tissue deflection profile along the vaginal axial direction observed at bladder inferior wall (FIG. 5(a)), the vesicovaginal connective tissue layers (FIG. 5(b)), and the rectovaginal connective tissue layers (FIG. 5(c)).

As shown in FIGS. 5(a)-(c), the change in the contour pattern indicated by gray scale levels starts from the vaginal introidal area and shifts towards the cervix (moving right to left in each figure), correlating to the device 10 insertion direction. The device 10 can simulate both tampon insertion and deployment processes by pushing the device 10 probe head 12 from the vaginal introitus towards the cervix. The design of the device 10 can facilitate prolonged strain energy, including predominantly at the bladder wall and vesicovaginal layers. The contour maps of bladder inferior wall (FIG. 5(a)) and vesicovaginal tissue layers (FIG. 5(b)) each show distinct changes of tissue deflection sustained up to the last phase of insertion process (at time equal to about 3.5 seconds). For the bladder wall and vesicovaginal layers the change in the contour map occurs early at the introitus and the change propagates through the mid vagina and cervix. Some of the strain energy has been attenuated at the vesicovaginal tissue before it actually reaches at the bladder wall. Rectovaginal connective tissue also shows those strain energy propagation (FIG. 5(c)), however, the depth of tissue layers impacted by the strain energy is limited, compared to the other two tissue layers such as bladder wall and vesicovaginal tissues.

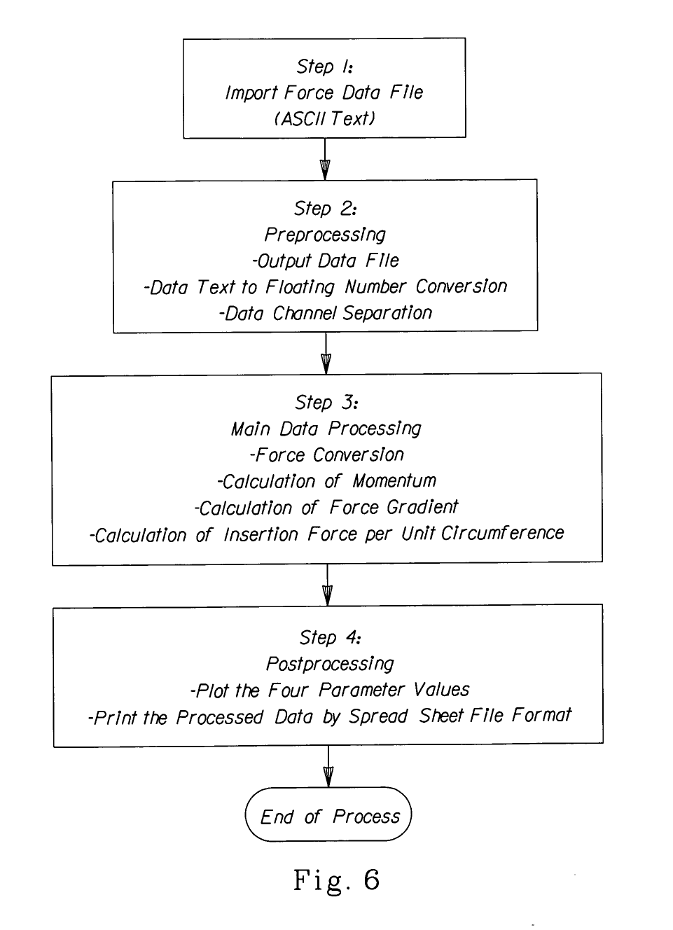

The current invention permits forces exerted on internal tissues to be measured in vivo. Signal processing of raw force profiles can provide additional insight on the physical deformation of tissues due to interaction with device 10. One embodiment of a method of force signal processing is shown in diagrammatic flow chart in FIG. 6. The original data format of a force signal can be in any format, but in one embodiment the data is in ASCII (American Standard Code for Information Interchange) which is a convenient format for manipulating by MatLab commands. Force data is imported in Step 1 and converted into a numeric format-floating point number in Step 2. Data channels are separated for time base and force profile data in Step 2 as well. Once a force profile with time base is ready, in Step 3 the Matlab program calculates the force profile, typically in units of gram force. Depending on the product application for which modeling by the present invention is employed, other physical properties can be processed. For example, if tampon applicators are the product of interest, different physical quantities such as momentum (considering the “mechanical” collision between the insertion device especially the tip and vaginal lumen), temporal force gradient or contact force along the circumference that makes direct contact with vaginal wall and introitus could be calculated and modeled to provide a more meaningful indicator of insertion deployment experience.

Change in momentum is simply calculated from the force (load) profile, F, by the formula:
∫F(t)dt[unit of gram—force sec].
The force gradient profile is also obtained by the formula:

$$\frac{ⅆ F ( t )}{ⅆ t} {\vert}_{max} [ unit of gram \text{-} force sec ] .$$

As the final step (Step 4), all data are saved in a suitable spread sheet file as is known in the art, and the data can be plotted or printed for further visualization and evaluation.

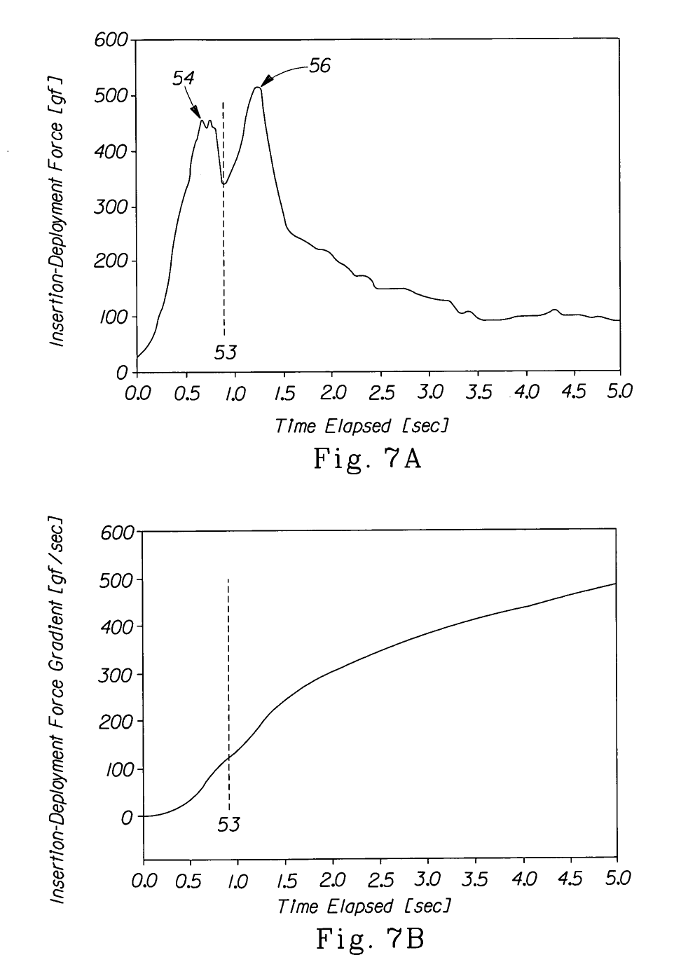

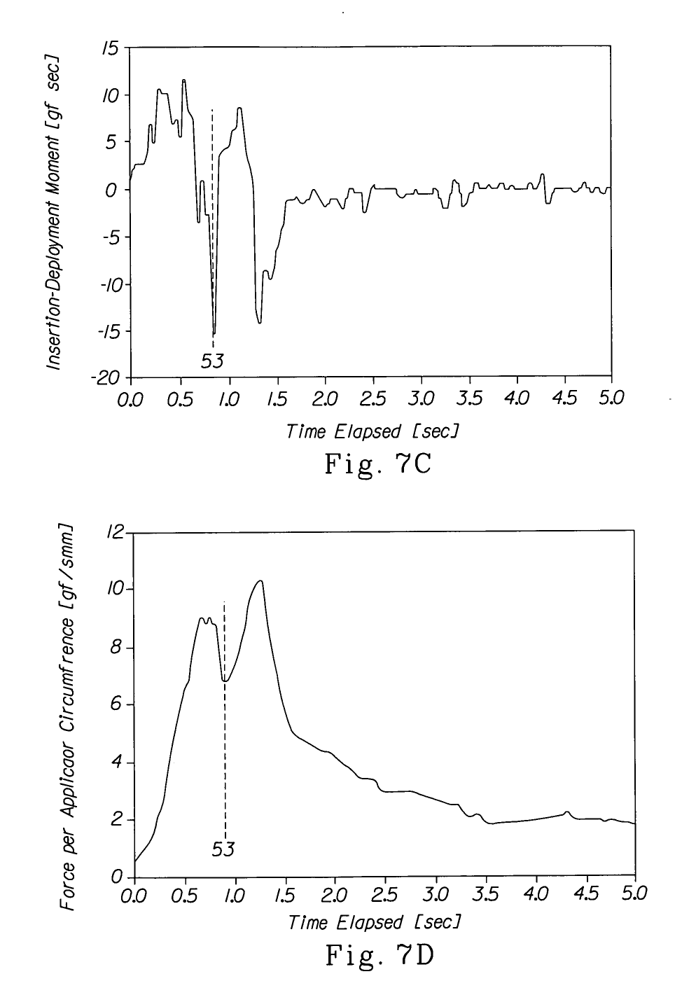

One example of the processed results is shown in the graphs of FIG. 7 showing the time dependent changes in force, momentum, temporal force gradient, and contact force for insertion of device 10 into a human vagina. The process involved in this in vivo test of the current invention involved both insertion and deployment-simulating the secondary insertion for deployment of device 10. The plot shown in FIG. 7(a) shows the force profile. Temporal position 53 demarcates insertion and deployment processes. Insertion force peak 54 and deployment force peak 56 are well discernible; the deployment process is additional to the insertion process. The plot shown in FIG. 7(b) shows change in momentum profile calculated over both processes; the momentum change increases monotonically. Temporal force gradient profile is shown at plot FIG. 7(c); rapid insertion process is depicted as high magnitude of force gradient for short time frame at the terminal of insertion process. Momentary resting period in two processes are also visible by two negative peaks at the terminal stage of both processes. The plot shown at FIG. 7(d) shows the contact force history, which is same profile as shown in FIG. 7(a) of force, but different magnitude scale.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for determining material properties of internal tissues of a body, the method comprising the steps of:
j. providing force measuring device, said force measuring device comprising:
i. a probe head having a dimension suitable for insertion into the body cavity;
ii. a load cell operatively connected to said probe head such that forces on said probe head are transmitted to said load cell;
iii. a relatively rigid portion joined to said probe head by a relatively flexible portion such that said probe head is flexibly joined to said relatively rigid portion and can move laterally with respect to a longitudinal center line of said relatively rigid portion; and
k. providing an imaging means for acquiring images of deformation or displacement of tissues inside a body cavity;
l. providing a calculation means for quantifying displacement of internal tissues;
m. inserting at least a portion of said probe head into the body cavity;
n. applying force with said probe head against an internal body tissue;
o. measuring said force applied to said internal body tissue;
p. acquiring images of displacement of said internal body tissue;
q. calculating said displacement of said internal body tissue; and
r. calculating material properties from said measured force and said calculated displacement.

2. The method for determining material properties of internal tissues of claim 1, wherein said load cell is joined to said relatively rigid portion.

3. The method for determining material properties of internal tissues claim 1, wherein said load cell is joined to said probe head.

4. The method for determining material properties of internal tissues of claim 1, wherein said load cell is disposed inside said probe head.

5. The method for determining material properties of internal tissues of claim 1, wherein said relatively flexible portion comprises a plastic tube.

6. The method for determining material properties of internal tissues of claim 1, wherein said force measuring device further comprises a handle portion.

7. The method for determining material properties of internal tissues of claim 6, wherein said load cell is joined to said relatively rigid portion and said handle.

8. The method for determining material properties of internal tissues claim 1, wherein said probe head comprises a material having a specific gravity of from about 0.98 to about 1.2.

9. The method for determining material properties of internal tissues claim 1, wherein said

10. The force measuring device of claim 1, wherein said imaging means comprises a device selected form the group consisting of: ultrasound, MRI, and X-ray.

11. A method for determining material properties of internal tissues of a body, the method comprising the steps of:
a. providing force measuring device, said force measuring device comprising:
i. a probe head having a dimension suitable for insertion into the body cavity;
ii. a load cell operatively connected to said probe head such that forces on said probe head are transmitted to said load cell;
iii. a relatively rigid portion joined to said probe head by a relatively flexible portion such that said probe head is flexibly joined to said relatively rigid portion and can move laterally with respect to a longitudinal center line of said relatively rigid portion; and
b. providing an imaging means for acquiring images of deformation or displacement of tissues inside a body cavity;
c. providing a calculation means for quantifying displacement of internal tissues;
d. providing numerical modeling software for performing finite element analysis;
e. inserting at least a portion of said probe head into the body cavity;
f. applying force with said probe head against an internal body tissue;
g. measuring said force applied to said internal body tissue;
h. acquiring images of displacement of said internal body tissue;
i. calculating said displacement of said internal body tissue; and
j. calculating material properties from said measured force and said calculated displacement,
k. constructing a numerical model for said probe;
l. constructing a numerical model for said internal tissues;
m. integrating said numerical model of said probe and said numerical model of said internal tissues and numerically simulating movement of said probe in said body cavity;
n. comparing simulated displacement of said internally tissues and simulated force applied to said probe from step (m) with quantified force from step (g) and displacement from step (i);
o. iterating steps (k) through (n) changing modeling variables as necessary until simulated values for displacement and force substantially agree with actual measured values.

12. The method for determining material properties of internal tissues of claim 11, wherein said load cell is joined to said relatively rigid portion.

13. The method for determining material properties of internal tissues claim 11, wherein said load cell is joined to said probe head.

14. The method for determining material properties of internal tissues of claim 11, wherein said load cell is disposed inside said probe head.

15. The method for determining material properties of internal tissues of claim 11, wherein said relatively flexible portion comprises a plastic tube.

16. The method for determining material properties of internal tissues of claim 11, wherein said force measuring device further comprises a handle portion.

17. The method for determining material properties of internal tissues of claim 16, wherein said load cell is joined to said relatively rigid portion and said handle.

18. The method for determining material properties of internal tissues claim 11, wherein said probe head comprises a material having a specific gravity of from about 0.98 to about 1.2.

19. The method for determining material properties of internal tissues of claim 11, wherein said imaging means comprises a device selected form the group consisting of: ultrasound, MRI, and X-ray.

20. The method for determining material properties of internal tissues of claim 11, wherein said internal cavity is a vagina.
